(19) United States
(12) Patent Application Publication
(10) Pub. No.: US 2002/0114806 A1
(43) Pub. Date: Aug. 22, 2002

(54) Uses of mammalian genes and related reagents

(76) Inventors: Annie Pardo-Semo, Coyoacan (MX); Moises Selman-Lama, Coyoacan (MX); Theodore R. Sana, Santa Clara, CA (US); Kathleen M. Smith, Palo Alto, CA (US); Robert L. Coffman, Portola Valley, CA (US); Albert Zlotnik, Palo Alto, CA (US)

Correspondence Address: DNAX RESEARCH INSTITUTE, 901 California Avenue, Palo Alto, CA, 94304-1104, US

(21) Appl. No.: 09/949,781

(22) Filed: Sep. 10, 2001

Related U.S. Application Data
(60) Provisional application No. 60/232,328, filed on Sep. 13, 2000.

(51) Int. Cl. A61K 39/395; C12Q 1/68; G01N 33/53; G01N 33/567

(52) U.S. Cl. 424/146.1; 435/6; 435/7.2

(57) ABSTRACT

Methods for treating, diagnosing, or evaluating mucosal surface medical conditions. Correlations of chemokine or receptor expression with gastrointestinal status are provided.

This filing claims benefit of U.S. Provisional Patent Application No. 60/232,328, filed Sep. 13, 2000.

### FIELD OF THE INVENTION

The present invention relates generally to uses of mammalian genes and related reagents. More specifically, the invention relates to identification of mammalian genes whose expression levels are implicated in medical conditions affecting mucosal surfaces, e.g., lung conditions. Diagnostic and therapeutic uses result.

### BACKGROUND OF THE INVENTION

Because inflammatory responses are often mediated by cytokine or chemokine activity, methods to evaluate synthesis of these signaling molecules would be advantageous for diagnosis of selected diseases. The present invention relates generally to identification of genes which may directly be of use to treat, or alternatively, to evaluate status of medical conditions affecting mucosal surfaces. See, e.g., Ogra, et al. (eds. 1999) Mucosal Immunity (2d ed.) Academic Press. The major mucosal surfaces include the gastrointestinal tract, the lungs and associated surfaces, and the female reproductive tract.

The severe complications of problems with mucosal surfaces can be seriously debilitating, and eventually may lead to death. Thus, a need exists for effective treatment, both prophylactic and curative, to alleviate the symptoms of those conditions. Alternatively, methods of diagnosis, e.g., of abnormal or modified health of those tissues will be useful. The present invention provides both.

### SUMMARY OF THE INVENTION

The present invention is based, in part, upon the observation that CCL18 expression correlates with various identified pulmonary conditions. In particular, the invention provides methods of diagnosing or evaluating a pulmonary condition comprising evaluating expression of CCL18. In various embodiments, the pulmonary condition is inflammatory or asthmatic, e.g., hypersensitivity pneumonitis, idiopathic pulmonary fibrosis, or asthma. Often, the evaluation is of nucleic acid or protein, e.g., histological. In other embodiments, the diagnosis is in combination with another diagnostic for the pulmonary condition.

The invention also provides methods of treating an inflammatory lung condition comprising blocking signaling mediated by CCL18. In preferred embodiments, the lung condition exhibits signs or symptoms of hypersensitivity pneumonitis, idiopathic pulmonary fibrosis, or asthma. Often the blocking is by administering an antibody which neutralizes CCL18 signaling, or by receptor desensitization. Often, the method will be used in combination with another treatment for the lung condition.

### DETAILED DESCRIPTION OF THE PREFERRED EMBODIMENTS

Outline

I. General

A. Lungs

B. Lung conditions and inflammation

C. Chemokines and Receptors

II. Antagonists

A. Blocking ligand

B. Blocking receptor

III. Diagnostic uses; Therapeutic compositions, methods

A. indications

B. combination compositions

C. unit dose

D. administration

I. General

Lungs and lung surfaces are very important since they serve a critical physiological function, e.g., respiration. Pulmonary ventilation and function depend on proper aspects of airway clearance and surface physics. See, e.g., Murray and Nadel (2000) Textbook of Respiratory Medicine Saunders (ISBN: 0721677118); Levitzky (1999) Pulmonary Physiology McGraw-Hill (ISBN: 0071345434); West (1998) Pulmonary Pathophysiology: The Essentials Lippincott (ISBN: 0683302256); Isenberg and Shapiro (eds. 1998) Respiratory Pathology and Pharmacotherapy Birkhauser, Boston; Kradin and Robinson (1996) Immunopathology of Lung Disease Buttersworth, Boston; Hasleton (ed. 1996) Spencer's Pathology of the Lung McGraw Hill (ISBN: 0071054480); and Cotes and Leathart (1993) Lung Function: Assessment and Application in Medicine Blackwell (ISBN: 0632035269). Inflammatory and other processes which affect them are likely to involve the chemokine signaling mechanisms. See, e.g., Nelson and Martin (eds. 2000) Cytokines in Pulmonary Disease Dekker.

The present invention resulted from studies directed to whether modified expression of chemokines or chemokine receptors correlated with conditions affecting medical conditions affecting the lung. Increased expression of chemokines could result in recruitment of inflammatory cells, e.g., macrophages, dendritic cells, or lymphocytes, and which may contribute to problems in lung inflammation and related conditions. A set of genes with no known disease associations in pulmonary conditions were selected to analyze on a cDNA panel. These included several chemokines and chemokine receptors.

Hypersensitivity Pneumonitis (HP) is an inflammatory disorder characterized by increased traffic and accumulation of T-lymphocytes in lung parenchyma. The mechanisms implicated in this process remain undefined. Here, lung expression was examined of DC-CK1/CCL18, a chemokine putatively involved in naive T cell recruitment, in patients with HP. Normal lung tissues and asthmatic airways were used as controls. CCL18 was measured by real-time quantitative PCR (e.g., TaqMan), and localized in the lung tissues by in situ hybridization and immunohistochemistry. The results indicate a significant induction (about 4 fold) of CCL18 expression in HP tissues when compared with both control lungs or asthmatic lungs. Reactive alveolar epithelial cells and interstitial macrophages were the main source of this chemokine. Importantly, there was a direct correlation between the levels of CCL18 detected in a given lung biopsy and the number of lymphocytes present in the bronchoalveolar lavage (BAL) fluid of that particular patient. High levels of CCL18 were observed in lungs of patients in an acute rather than chronic phase of HP. These findings suggest a role for CCL18 in the pathogenesis of HP.

Hypersensitivity Pneumonitis (HP) comprises a group of diffuse inflammatory disorders of the lung parenchyma provoked by exposure to a variety of organic particles, and characterized by lymphocytic alveolitis. Selman (1998) in Schwarz (ed.) Interstitial Lung Disease Decker, Hamilton, Ontario. One of the most frequent forms of HP is the so-called pigeon breeder's disease, which is induced by the inhalation of avian antigens. Perez-Padilla, et al. (1993) Am. Rev. Respir. Dis. 148:49-53. In general, HP represents a serious lung disorder where approximately thirty percent of patients with subacute/chronic disease evolve into a fibrotic disease which is usually fatal. Perez-Padilla, et al. (1993) Am. Rev. Respir. Dis. 148:49-53; and Pardo, et al. (2000) Am. J. Respir. Crit. Care Med. 161:1698-1704. While significant progress has been made recently in our understanding of the pathology of this disease (see Denis (1995) Am. J. Respir. Crit. Care Med. 151:164-169; Trentin, et al. (1990) J. Immunol. 145:2147-2154; and Selman, et al. (1993) Semin. Respir. Med. 14:353.), the mechanisms responsible for lymphocyte recruitment in this ailment remain to be elucidated.

The chemokines are a superfamily of small, secreted proteins that regulate leukocyte migration. Many new members of this superfamily have been described in the last few years. See Zlotnik and Yoshie (2000) Immunity 12:121-127. This family of molecules may represent one of the first molecular superfamilies where most of the members are known. Several chemokines have been associated with various diseases. See, e.g., Rossi and Zlotnik (2000) Annu. Rev. Immunol. 18:217-242; and Homey, et al. (2000) J. Immunol. 164:6621-6632. A number of chemokines have been reported to be expressed in the lung, including IL-8/CXCL8, eotaxin/CCL11, MIP-3α/CCL20, and DC-CK1/PARC/AMAC-1/CCL18. Rossi and Zlotnik (2000) Annu. Rev. Immunol. 18:217-242. Interestingly, there is even one reported chemokine that is specifically expressed in the lung, lungkine/CXCL15. Rossi, et al. (1999) J. Immunol. 162:5490-5497. In this report we will use the new chemokine nomenclature recently proposed. Zlotnik and Yoshie (2000) Immunity 12:121-127.

We hypothesized that chemokines may play a critical role in HP. To test this hypothesis, we performed a comprehensive analysis of the expression of chemokines and their receptors in HP using real time PCR via TaqMan. See Homey, et al. (2000) J. Immunol. 164:6621-6632. The results indicate that one chemokine in particular, CCL18, is associated with the development of HP. CCL18 was originally reported as a dendritic cell product (Adema, et al. (1997) Nature 387:713-717) but was subsequently shown to be produced by monocytes induced by IL-4 (Kodelja, et al. (1998) J. Immunol. 160:1411-1418). Another report described that CCL18 is strongly expressed in the lung and was therefore named PARC (pulmonary and activation related chemokine) by this group. Hieshima, et al. (1997) J. Immunol. 159:1140-1149. However, the role of this chemokine in human lung diseases remains unexplored. Here, we report that increased CCL18 expression is associated with HP, and may represent an important mediator in the pathology of this disease.

II. Antagonists

Blockage of the signaling pathway can be achieved by antagonists of the chemokine, e.g., antibodies to the ligand, antibodies to the receptor, etc. Interference with the ligand-receptor interaction has proven to be an effective strategy for the development of antagonists, including small molecule antagonists. In addition, receptor desensitization may be effected, e.g., by administration of ligand.

There are various means to antagonize the signaling mediated by ligand. Two apparent means are to block the ligand with antibodies; a second is to block the receptor with antibodies. Various epitopes should exist on each which will block their interaction, e.g., causing steric hindrance blocking interaction. The correlation of ability to block signaling would not necessarily be expected to correlate with binding affinity to either ligand or receptors. Steric effects may be much more important in which antibodies would be effective antagonists.

Alternatively, small molecule libraries may be screened for compounds which may block the interaction or signaling mediated by an identified ligand-receptor pairing.

The present invention provides for the use of an antibody or binding composition which specifically binds to a specified chemokine ligand, preferably mammalian, e.g., primate or human, or other. Antibodies can be raised to various chemokine proteins, including individual, polymorphic, allelic, strain, or species variants, and fragments thereof, both in their naturally occurring (full-length) forms or in their recombinant forms. Additionally, antibodies can be raised to receptor proteins in both their native (or active) forms or in their inactive, e.g., denatured, forms. Anti-idiotypic antibodies may also be used.

A number of immunogens may be selected to produce antibodies specifically reactive with ligand or receptor proteins. Recombinant protein is a preferred immunogen for the production of monoclonal or polyclonal antibodies. Naturally occurring protein, from appropriate sources, e.g., primate, etc., may also be used either in pure or impure form. Synthetic peptides, made using the appropriate protein sequences, may also be used as an immunogen for the production of antibodies. Recombinant protein can be expressed and purified in eukaryotic or prokaryotic cells as described, e.g., in Coligan, et al. (eds. 1995 and periodic supplements) Current Protocols in Protein Science John Wiley & Sons, New York, N.Y.; and Ausubel, et al (eds. 1987 and periodic supplements) Current Protocols in Molecular Biology, Greene/Wiley, New York, N.Y. Naturally folded or denatured material can be used, as appropriate, for producing antibodies. Either monoclonal or polyclonal antibodies may be generated, e.g., for subsequent use in immunoassays to measure the protein, or for immunopurification methods. Methods also exist for induction of quasi-autoimmune responses to specific antigens.

Methods of producing polyclonal antibodies are well known to those of skill in the art. Typically, an immunogen, preferably a purified protein, is mixed with an adjuvant and animals are immunized with the mixture. The animal's immune response to the immunogen preparation is monitored by taking test bleeds and determining the titer of reactivity to the protein of interest. For example, when appropriately high titers of antibody to the immunogen are obtained, usually after repeated immunizations, blood is collected from the animal and antisera are prepared. Further fractionation of the antisera to enrich for antibodies reactive to the protein can be done if desired. See, e.g., Harlow and Lane; or Coligan. Immunization can also be performed through other methods, e.g., DNA vector immunization. See, e.g., Wang, et al. (1997) Virology 228:278-284.

Monoclonal antibodies may be obtained by various techniques familiar to researchers skilled in the art. Typically, spleen cells from an animal immunized with a desired antigen are immortalized, commonly by fusion with a myeloma cell. See, Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds. 1994 and periodic supplements) Cell and Tissue Culture: Laboratory Procedures, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Antibodies or binding compositions, including binding fragments and single chain versions, against predetermined fragments of ligand or receptor proteins can be raised by immunization of animals with conjugates of the fragments with carrier proteins. Monoclonal antibodies are prepared from cells secreting the desired antibody. These antibodies can be screened for binding to normal or defective protein. These monoclonal antibodies will usually bind with at least a KD of about 1 mM, more usually at least about 300 μM, typically at least about 10 μM, more typically at least about 30 μM, preferably at least about 10 μM, and more preferably at least about 3 μM or better.

In some instances, it is desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) Basic and Clinical Immunology (4th ed.) Lange Medical Publications, Los Altos, Calif., and references cited therein; Harlow and Lane (1988) Antibodies: A Laboratory Manual CSH Press; Goding (1986) Monoclonal Antibodies: Principles and Practice (2d ed.) Academic Press, New York, N.Y.; and particularly in Kohler and Milstein (1975) Nature 256:495-497, which discusses one method of generating monoclonal antibodies. Summarized briefly, this method involves injecting an animal with an immunogen. The animal is then sacrificed and cells taken from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Other suitable techniques involve selection of libraries of antibodies in phage or similar vectors. See, e.g., Huse, et al. (1989) “Generation of a Large Combinatorial Library of the Immunoglobulin Repertoire in Phage Lambda,” Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see, Cabilly, U.S. Pat. No. 4,816,567; and Queen, et al. (1989) Proc. Nat'l Acad. Sci. USA 86:10029-10033; or made in transgenic mice, see Mendez, et al. (1997) Nature Genetics 15:146-156; also see Abgenix and Medarex technologies.

Antibodies are merely one form of specific binding compositions. Other binding compositions, which will often have similar uses, include molecules that bind with specificity to ligand or receptor, e.g., in a binding partner-binding partner fashion, an antibody-antigen interaction, or in a natural physiologically relevant protein-protein interaction, either covalent or non-covalent, e.g., proteins which specifically associate with desired protein. The molecule may be a polymer, or chemical reagent. A functional analog may be a protein with structural modifications, or may be a structurally unrelated molecule, e.g., which has a molecular shape which interacts with the appropriate binding determinants. Antibody binding compounds, including binding fragments, of this invention can have significant diagnostic or therapeutic value. They can be useful as non-neutralizing binding compounds and can be coupled to toxins or radionuclides so that when the binding compound binds to the antigen, a cell expressing it, e.g., on its surface, is killed. Further, these binding compounds can be conjugated to drugs or other therapeutic agents, either directly or indirectly by means of a linker, and may effect drug targeting.

III. Diagnostic Uses; Therapeutic Compositions, Methods

HP is a diffuse inflammatory disease characterized by the accumulation of T lymphocytes within the bronchoalveolar structures of the lung. Although the etiology of the disease is known, the sequence of pathogenic events leads to this state has not been clearly defined. Several mechanisms could be involved in the increased traffic and accumulation of T cells in the lung parenchyma. Chemokines have in recent years been recognized as critical mediators that regulate trafficking of various leukocyte populations to specific anatomical locations. See, e.g., Zlotnik and Yoshie (2000) Immunity 12:121-127. It is therefore reasonable to hypothesize that they may be involved in the recruitment of pathogenic T cells in HP. To test this hypothesis, we undertook a systematic analysis of the expression of chemokine ligands and their receptors in various human lung diseases. Of the 16 chemokines tested, the most dramatic association observed was between HP and the chemokine now known as CCL18 (Zlotnik and Yoshie (2000) Immunity 12:121-127), which was originally described as DC-CK1, PARC, or AMAC-1 (Adema, et al. (1997) Nature 387:713-717; Hieshima, et al. (1997) J. Immunol. 159:1140-1149; and Kodelja, et al. (1998) J. Immunol. 160:1411-1418). While CCL18 was initially believed to be a dendritic cell product (Adema, et al. (1997) Nature 387:713-717), its expression was subsequently reported to be induced in macrophages by various cytokines including IL4, IL-10, or IL-13 (Kodelja, et al. (1998) J. Immunol. 160:1411-1418). Importantly, it was also recognized in early studies that the expression of this chemokine was high in the lung. Hieshima, et al. (1997) J. Immunol. 159:1140-1149. However, no detailed analyses of the potential role of this chemokine in human lung diseases have been performed. It has, however, recently been reported that CCL18 is strongly expressed in the liver during hepatitis C infection. Kusano, et al. (2000) Lab. Invest. 80:415-422. CCL18 is known to act on some T cell subsets (Adema, et al. (1997) Nature 387:713-717; Kodelja, et al. (1998) J. Immunol. 160:1411-1418; and Hieshima, et al. (1997) J. Immunol. 159:1140-1149), and interestingly, it exists only in the human, not mouse. The latter observation is explained by the finding that CCL18 likely arose recently (in evolutionary terms) from the fusion of two MIP-1α/CCL3 genes. Tasaki, et al. (1999) Genomics 55:353-357. Despite its strong similarity to MIP-1α/CCL3, CCL18 does not bind CCR1, CCR3 or CCR5. Here we demonstrate that lungs from patients with subacute/chronic HP strongly over-express CCL18 when compared to normal lung parenchyma. CCL18 is produced primarily by reactive type 2 pneumocytes and interstitial macrophages, as shown by in situ hybridization and immunochemistry.

Interestingly, T-lymphocytes are also recruited to the airways of asthmatic patients. Umibe, et al. (2000) Clin. Exp. Immunol. 119:390-397; and Yssel and Groux (2000) Int. Arch. Allergy. Immunol. 121:10-18. In the asthmatic lung tissues studied here, the expression of CCL18 was similar to that observed in the normal lung parenchyma. However, asthma is a disease affecting the airways, not the lung parenchyma, which is mostly composed of alveoli. A possible explanation is that the tissue studied here, asthmatic lung parenchyma, is not affected by the asthmatic disease process. In addition, these tissues were obtained from individuals who died suddenly and were not necessarily having an asthma attack. Their lungs were rejected for transplants due to their history of asthma. The characteristics of the asthmatic tissues studied here indicate that the expression level of CCL18 is not increased in lung parenchyma in asthmatics in their basal state. However, this conclusion does not eliminate the possibility that CCL18 expression may be up-regulated once an asthmatic attack commences. This leaves open the possibility that CCL18 may play a role in T lymphocyte recruiting to asthmatic airways during the acute phase of the disease.

On the other hand, HP is believed to be a predominantly T helper type 1 lung disorder (Yamasaki, et al. (1999) J. Immunol. 163:3516-3523), while asthma is a Th2 type (Wills-Karp (1999) Annu. Rev. Immunol. 17:255-281) disease. This suggests that CCL18 may either regulate T cell function in the lung, or it may preferentially chemoattract Th1 cells. A variety of receptors for and responses towards chemokines have been reported to be differentially associated with Th subsets. For example, CCR5 is preferentially found on Th1 cells, whereas CCR3, CCR4, and CCR8 on Th2 cells (see Syrbe, et al. (1999) Springer Semin. Immunopathol. 21:263; and Zlotnik and Yoshie (2000) Immunity 12:121-127), suggesting that their ligands play a role in the preferential recruitment of Th1 or Th2-biased cells during the development and polarization of immune responses. In addition, the factors responsible for the polarization of specific immune responses into a predominant Th1 or Th2 profiles are not completely understood, and it is possible that CCL18 may even play a more direct role in the differentiation or development of immune responses.

The receptor for this chemokine has not yet been matched (Zlotnik and Yoshie (2000) Immunity 12:121-127), but these results strongly suggest that the lymphocytes found in the BAL of HP patients were chemoattracted to the lung by CCL18. Thus, these data suggest that BAL T cells express the CCL18 receptor. Finally, these observations suggest that inhibitors of the CCL18/receptor interaction will have therapeutic effects in HP.

The etiology and pathogenesis of these lung diseases are incompletely understood, but they cause significant morbidity in many patients. Collectively these studies suggest that antagonizing these chemokines or their receptors, with the appropriate entity may offer a therapeutic modality in these respiratory tract conditions, e.g., bronchitis, alveolitis, emphysema, asthma, interstitial lung diseases, chronic obstructive pulmonary disease (COPD; see, e.g., Murray (1996) Frontline Treatment of COPD), etc. See, e.g., Albert, et al. (1999) Comprehensive Respiratory Medicine Mosby; Fishman and Elias (eds. 1998) Fishman's Pulmonary Diseases and Disorders McGraw Hill; and Kradin (ed. 1996) Immunopathology of Lung Disease Buttersworth-Heinemann.

Diagnostic methods include such aspects as prediction of prognosis; definition of subsets of patients who will either respond or not respond to a particular therapeutic course; diagnosis of pulmonary conditions or diseases or subtypes of conditions or diseases; or assessing response to therapy.

Antagonists to chemokine mediated signaling have been implicated in a manner suggesting significant therapeutic effects, e.g., to decrease or prevent occurrence of symptoms. Small molecule antagonists for 7 transmembrane receptors and chemokine receptors are well known. Pertussis toxin can block the interaction of such receptors with the associated signaling G-protein coupled receptors.

The antagonists of the present invention can be administered alone or in combination with another inhibitor of the same or accompanying pathway; or other compounds used for the treatment of symptoms, e.g., antagonists, or steroids such as glucocorticoids.

In contrast, chemokines could be administered as an adjuvant for immunization, e.g., to trigger lung immunity. See, e.g., Ogra, et al. (eds. 1999) Mucosal Immunity (2d ed.) Academic Press.

To prepare pharmaceutical or sterile compositions including the antibody or binding composition thereof, the antibody or binding composition is typically admixed with a pharmaceutically acceptable carrier or excipient which is preferably inert. Preparation of such pharmaceutical compositions is known in the art, see, e.g., Remington's Pharmaceutical Sciences and U.S. Pharmacopeia: National Formulary, Mack Publishing Company, Easton, Pa. (1984).

Antibodies or binding compositions are normally administered parentally, preferably intravenously. Since such protein or peptide antagonists may be immunogenic they are preferably administered slowly, either by a conventional IV administration set or from a subcutaneous depot, e.g. as taught by Tomasi, et al, U.S. Pat. No. 4,732,863. Aerosolizers or inhalers may be used.

When administered parenterally the antibodies or fragments will be formulated in a unit dosage injectable form (solution, suspension, emulsion) in association with a pharmaceutically acceptable parenteral vehicle. Such vehicles are inherently nontoxic and nontherapeutic. The antagonist may be administered in aqueous vehicles such as water, saline, or buffered vehicles with or without various additives and/or diluting agents. Alternatively, a suspension, such as a zinc suspension, can be prepared to include the peptide. Such a suspension can be useful for subcutaneous (SQ) or intramuscular (IM) injection. The proportion of antagonist and additive can be varied over a broad range so long as both are present in effective amounts. The antibody is preferably formulated in purified form substantially free of aggregates, other proteins, endotoxins, and the like, at concentrations of about 5 to 30 mg/ml, preferably 10 to 20 mg/mi. Preferably, the endotoxin levels are less than 2.5 EU/ml. See, e.g., Avis, et al. (eds.)(1993) Pharmaceutical Dosage Forms: Parenteral Medications 2d ed., Dekker, NY; Lieberman, et al. (eds. 1990) Pharmaceutical Dosage Forms: Tablets 2d ed., Dekker, NY; Lieberman, et al. (eds. 1990) Pharmaceutical Dosage Forms: Disperse Systems Dekker, NY; Fodor, et al. (1991) Science 251:767-773, Coligan (ed.) Current Protocols in Immunology; Hood, et al. Immunology Benjamin/Cummings; Paul (ed.) Fundamental Immunology; Academic Press; Parce, et al. (1989) Science 246:243-247; Owicki, et al. (1990) Proc. Nat'l Acad. Sci. USA 87:4007-4011; and Blundell and Johnson (1976) Protein Crystallography, Academic Press, New York.

Selecting an administration regimen for an antagonist depends on several A factors, including the serum or tissue turnover rate of the antagonist, the level of symptoms, the immunogenicity of the antagonist, and the accessibility of the target cells. Preferably, an administration regimen maximizes the amount of antagonist delivered to the patient consistent with an acceptable level of side effects. Accordingly, the amount of antagonist delivered depends in part on the particular antagonist and the severity of the condition being treated. Guidance in selecting appropriate doses is found in the literature on therapeutic uses of antibodies, e.g. Bach et al., chapter 22, in Ferrone, et al. (eds. 1985) Handbook of Monoclonal Antibodies Noges Publications, Park Ridge, N.J.; and Haber, et al. (eds.) (1977) Antibodies in Human Diagnosis and Therapy, Raven Press, New York, N.Y. (Russell, pgs. 303-357, and Smith, et al., pgs. 365-389).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of the inflammation, e.g., level of inflammatory cytokines produced. Preferably, an antibody or binding composition thereof that will be used is derived from the same species as the animal targeted for treatment, thereby minimizing a humoral response to the reagent.

The total weekly dose ranges for antibodies or fragments thereof, which specifically bind to ligand or receptor range generally from about 10 μg, more generally from about 100 μg, typically from about 500 μg, more typically from about 1000 μg, preferably from about 5 mg, and more preferably from about 10 mg per kilogram body weight. Generally the range will be less than 100 mg, preferably less than about 50 mg, and more preferably less than about 25 mg per kilogram body weight.

The weekly dose ranges for antagonists of chemokine receptor mediated signaling, e.g., antibody or binding fragments, range from about 1 μg, preferably at least about 5 μg, and more preferably at least about 10 μg per kilogram of body weight. Generally, the range will be less than about 1000 μg, preferably less than about 500 μg, and more preferably less than about 100 μg per kilogram of body weight. Dosages are on a schedule which effects the desired treatment and can be periodic over shorter or longer term. In general, ranges will be from at least about 10 μg to about 50 mg, preferably about 100 μg to about 10 mg per kilogram body weight. Small molecule antagonists will typically have similar molar concentrations, but because they have smaller molecular weights, will have lesser weight doses.

The present invention also provides for administration of antibodies or binding compositions in combination with known therapies, e.g., steroids, particularly glucocorticoids, which alleviate the symptoms, e.g., associated with inflammation, or antibiotics or anti-infectives. Daily dosages for glucocorticoids will range from at least about 1 mg, generally at least about 2 mg, and preferably at least about 5 mg per day. Generally, the dosage will be less than about 100 mg, typically less than about 50 mg, preferably less than about 20 mg, and more preferably at least about 10 mg per day. In general, the ranges will be from at least about 1 mg to about 100 mg, preferably from about 2 mg to 50 mg per day. Suitable dose combinations with antibiotics, anti-infectives, or anti-inflammatories are also known.

The phrase “effective amount” means an amount sufficient to ameliorate a symptom or sign of the medical condition. Typical mammalian hosts will generally be primates, including humans. An effective amount for a particular patient may vary depending on factors such as the condition being treated, the overall health of the patient, the method route and dose of administration and the severity of side affects. When in combination, an effective amount is in ratio to a combination of components and the effect is not limited to individual components alone.

An effective amount of antagonist will decrease the symptoms typically by at least about 10%; usually by at least about 20%; preferably at least about 30%; or more preferably at least about 50%. The present invention provides reagents which will find use in therapeutic applications as described elsewhere herein, e.g., in the general description for treating disorders associated with the indications described, e.g., inflammatory conditions, chronic or acute, etc. See, e.g., Dayer (1999) J. Clin. Invest. 104:1337-1339; Gracie, et al. (1999) J. Clin. Invest. 104:1393-1401; Berkow (ed.) The Merck Manual of Diagnosis and Therapy, Merck & Co., Rahway, N.J.; Thorn, et al. Harrison's Principles of Internal Medicine, McGraw-Hill, NY; Gilman, et al. (eds.) (1990) Goodman and Gilman's: The Pharmacological Bases of Therapeutics, 8th Ed., Pergamon Press; Remington's Pharmaceutical Sciences, 17th ed. (1990), Mack Publishing Co., Easton, Pa.; Langer (1990) Science 249:1527-1533; Merck Index, Merck & Co., Rahway, N.J.; and Physician's Desk Reference (PDR).

The broad scope of this invention is best understood with reference to the following examples, which are not intended to limit the inventions to the specific embodiments.

### EXAMPLES

I. General Methods

Some of the standard methods are described or referenced, e.g., in Maniatis, et al. (1982) Molecular Cloning. A Laboratory Manual, Cold Spring Harbor Laboratory, Cold Spring Harbor Press; Sambrook, et al. (1989) Molecular Cloning: A Laboratory Manual, (2d ed.), vols. 1-3, CSH Press, NY; Ausubel, et al., Biology, Greene Publishing Associates, Brooklyn, N.Y.; or Ausubel, et al. (1987 and Supplements) Current Protocols in Molecular Biology, Greene/Wiley, New York. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification” in Meth. Enzymol., vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1990) “Purification of Recombinant Proteins with Metal Chelate Absorbent” in Setlow (ed.) Genetic Engineering, Principle and Methods 12:87-98, Plenum Press, N.Y.; and Crowe, et al. (1992) QIAexpress: The High Level Expression & Protein Purification System QIAGEN, Inc., Chatsworth, Calif.

Computer sequence analysis is performed, e.g., using available software programs, including those from the GCG (U. Wisconsin) and GenBank sources. Public sequence databases were also used, e.g., from GenBank and others.

II. Patient Evaluation

Study Population

Nonsmoking female patients with sub-acute/chronic hypersensitivity pneumonitis (Pardo, et al. (2000) Am. J. Respir. Crit. Care Med. 161:1698-1704.) were studied. This study was approved by the Ethics Committee of the Institute (INER), and informed written consent was obtained from each subject. Diagnosis of HP was obtained according to international criteria including: 1) pigeon exposure preceding disease and positive serum antibodies against avian antigens; 2) shortness of breath with partial improvement upon avoidance of the avian antigen exposure; 3) clinical, radiological, and functional features of an interstitial lung disease (ILD); 4) more than 40% lymphocytes in bronchoalveolar lavage (BAL) fluid; and 5) lung histology compatible with HP (Selman (1998) in Schwarz (ed.) Interstitial Lung Disease Decker, Hamilton, Ontario; Perez-Padilla, et al. (1993) Am. Rev. Respir. Dis. 148:49-53; and Pardo, et al. (2000) Am. J. Respir. Crit. Care Med. 161:1698-1704) Briefly, the tissue samples showed diffuse interstitial inflammation of mononuclear predominance, mainly lymphocytes, and frequent multinucleated giant cells in terminal and respiratory bronchioles, as well as in the alveolar walls. Small and loosely arranged granulomas were observed in the interstitium. Biopsy cultures were negative for bacteria, mycobacteria, and fungi.

Lung samples were taken from hypersensitivity pneumonitis and idiopathic pulmonary fibrosis (IPF) patients by open lung biopsy usually one week after hospital admission. None of the patients had been treated with steroids or immunosuppressive drugs at the time of biopsy. A portion of the biopsy was immediately frozen in liquid nitrogen for RNA extraction and subsequent expression analysis by TaqMan.

For quantitative PCR analyses, control lung tissues were lung parenchyma from transplant donors, autopsy donors, or normal adjacent tissue from patients undergoing surgery for lung cancer. The available clinical history of these patients indicated that they had no known infections or lung diseases. One donor had a history of smoking. In addition, two normal lung RNAs were purchased from Clontech (Palo Alto, Calif.). One was from a single individual and the other was from a pool of RNA from five individuals. The Clontech pool had lung RNA from both males and females, ranging in age from 14 to 40 years old. In addition, lung tissue from nine asthmatic transplant donors (5 females and 4 males, range 4 to 71 years) was also studied. Control lung tissues for PCR analyses were obtained from the National Disease Research Interchange (Philadelphia, Pa.). For immunohistochemistry and in situ hybridization studies, control lung tissue samples were obtained from autopsies of patients who died from non-lung causes. Selected lung fragments, which appeared macroscopically and microscopically normal, were used.

III. Lymphocyte Numbers in Bronchoalveolar Lavage (BAL)

BAL fluids were spun down (cytospin), and stained for differential leukocyte counts. Samples were counted in a double blind fashion.

IV. RNA Preparation and PCR Analysis

RNA was extracted from lung tissue either using guanidinium thiocyanate followed by centrifugation in cesium chloride (Sambrook et. al 1989) or RNA STAT-60 (Tel-Test). RNA quality was assessed by agarose gel electrophoresis. Five μg of total RNA was treated with RNAse-free DNAse I (Boehringer Mannheim) in First Strand Synthesis Buffer in the presence of RNAsin (Promega). Samples were incubated for 20 min at 37° C., heated for 10 min at 70° C., and then immediately chilled on ice. A mixture of 2.5 μg of oligo d(T)₁₂₋₁₅ (Boehringer Mannheim) and 250 ng of random hexamers (Promega) was added to each sample. Samples were heated to 70° C. for 10 min, rapidly chilled on ice, and then briefly spun in a microfuge. cDNA was generated from the RNA using Superscript II reverse transcriptase (GIBCO-BRL) according to manufacturer's instructions in a final volume of 100 μl.

10 ng of cDNA/reaction was analyzed for expression of CCL18 and ubiquitin on a GeneAmp 5700 Sequence Detector (PE Applied Biosystems) in a 25 μl reaction. CCL18 was detected using primers and probe (PE Applied Biosystems) with TaqMan Universal Master Mix (PE Applied Biosystems) or primers alone and SYBR Green PCR Master Mix (PE Applied Biosystems). Ubiquitin was detected using appropriate 200 nM primers with SYBR Green PCR Mater Mix. The data was analyzed to calculate a cycle threshold value (Ct) for each sample with GeneAmp 5700 SDS Software (PE Applied Biosystems). Samples were assayed three times for CCL18 and twice for ubiquitin and the average of the reading for both genes was used to calculate the relative level of CCL18 mRNA in the tissue using the following formula: 2^{(Ct of ubiquitin-Ct of CCL18)}*10,000 for each sample. Mean and standard errors was calculated for each group.

V. Statistical Analysis

Statistical analysis was performed with JMP 3.2.2 (SAS Institute, Inc.). The TaqMan data for the relative level of CCL18 was log transformed and a one-way ANOVA performed. The log transformed CCL18 level was used as the dependent variable and the disease group was used as the independent variable. All pair wise comparisons were made using the Tukey-Kramer HSD test with α=0.01. This analysis demonstrated that both the HP and the IPF groups were different from the control group, and, additionally both were different from the asthmatic group. We also examined the correlation of the level of CCL18 with the percentage of lymphocytes present in the BAL. A positive correlation was observed.

VI. Immunohistochemistry

Tissue sections were deparaffinized, rehydrated, and then blocked with 3% H₂O₂ in methanol for 30 min followed byantigen retrieval performed with citrate buffer 10 mM pH 6.0 for 5 min in a microwave. Tissue sections were then incubated with an antibody diluent with background reducing components (Dako, Carpinteria, Calif.) diluted 1/100 in PBS for 45 min. Antibodies were applied and incubated at 4° C. overnight. A secondary biotinylated anti-immunoglobulin followed by horseradish peroxidase-conjugated streptavidin (BioGenex, San Ramon Calif.) was used according to manufacturer's instructions. 3-amino-9-ethyl-carbazole (AEC, BioGenex) in acetate buffer containing 0.05% H₂O₂ was used as substrate. See Pardo, et al. (2000) Am. J. Respir. Crit. Care Med. 161:1698-1704; and Umibe, et al. (2000) Clin. Exp. Immunol. 119:390-397. The sections were counterstained with hematoxylin. The primary antibody was replaced by non-immune serum for negative control slides.

VII. Monoclonal Antibody Production

Mouse anti-CCL18 monoclonal antibodies were produced in BALB/c mice. Mice were immunized i.p. with 25 μg of CCL18/lg fusion protein emulsified in complete Freund's adjuvant (CFA), and then boosted every two to three weeks with 15 μg of the same protein in incomplete Freund's adjuvant (IFA). The final boost was performed with cleaved CCL18. Splenocytes from immunized animal were fused with mouse myeloma SP2/0. Hybridoma supernatants were screened by ELISA on cleaved CCL18 coated plates and by Western Blot analysis. Positive producing hybridomas were cloned and re-screened.

VIII. In Situ Hybridization

Riboprobes for in situ hybridization were generated from human cDNA CCL18 cloned into pSPORT1 (Gibco/BRL). The plasmid was linearized before translation with Kpn I. An antisense 628 bp fragment was transcribed with T7, and a sense 150 bp fragment with SP6 RNA polymerases respectively. The transcription of sense and antisense transcripts was performed with a labeling mixture containing digoxigenin-UTP (Boehringer Mannheim, Mannheim, Germany).

In situ hybridization was performed on 4 μm sections. See, e.g., Pardo, et al. (1998) Am. J. Pathol. 153:833-844; and Perez-Ramos, et al. (1 999) Am. J. Respir Crit. Care Med. 160:1274-1282. Briefly, the sections mounted on silanized slides were incubated in 0.001% proteinase K (Sigma Chemical Co., St. Louis Mo.) for 20 min at 37° C. After acetylation with acetic anhydride the sections were prehybridized for 1 h at 45° C. in a hybridization buffer. The sections were incubated with the digoxigenin-labeled probes at 45° C overnight. Some sections were hybridized with digoxigenin-labeled sense RNA probe. The tissues were incubated with a polyclonal sheep anti-digoxigenin antibody coupled to alkaline phosphatase (Boehringer Mannheim Co, Indianapolis Ind.) for 1 h at room temperature. The color reaction was performed by incubation with Fast Red chromogen (Biomeda Corp, Foster City Calif.). Sections were lightly counter-stained with hematoxylin.

IX. Patient Characteristics

Baseline characteristics of the patients with hypersensitivity pneumonitis are summarized in Table 1. All patients showed clinical and functional evidence of interstitial lung disease, with variable degrees of dyspnea, decreased lung capacities, and hypoxemia at rest that worsens during exercise. Differential cell counts in bronchoalveolar fluid were characterized by marked lymphocytosis, usually well over 50%.

TABLE 1
BASELINE CHARACTERISTICS OF THE
INITIAL STUDY POPULATION

| | |
|---|---|
| Number of patients | 10 |
| Age (years) | 51 ± 16 |
| Time elapsed to first visit (months) | 23 ± 20 |
| Dyspnea score* | 2.3 ± 0.6 |
| FVC % p** | 55 ± 19 |
| TLC % p** | 63 ± 12 |
| PaO₂ mm Hg | 49.5 ± 9 |
| BAL lymphocytes | 70 ± 20 |
| BAL macrophages | 28 ± 19 |
| BAL neutrophils | 0.5 ± 1 |
| BAL eosinophils | 1.7 ± 2 |

*Dyspnea score: dyspnea at exercise, 1: slight; 2: moderate; 3: severe; 4: dyspnea at rest.
**Percent of predicted.

X. Quantification of CCL18

To determine whether any chemokines were over-expressed in lung tissue from hypersensitivity pneumonitis patients, we assayed cDNA prepared from RNA extracted from control and diseased lungs, for the level a panel of chemokines or their receptors using real-time PCR (TaqMan). Included on the panel were cDNAs made from 20 control lung samples, nine asthmatic lung samples, nine idiopathic pulmonary fibrosis biopsies, and ten hypersensitivity pneumonitis biopsies taken at the time of diagnosis. The initial series of analyses included the following chemokines and receptors: 1-309/CCL1, MCP-1/CCL2, MIP-1α/CCL3, MIP-1β/CCL4, RANTES/CCL5, MCP-3/CCL7, eotaxin/CCL11, TARC/CCL17, DC-CK1/CCL18, MIP-3β/CCL19, MIP-3α/CCL20, MDC/CCL22, VIC/CCL28, ENA-78/CXCL5, IL-8/CXCL8, lymphotactin/XCL1, CCR3, CCR4, CCR5, CCR6, CCR7, CCR8, CXCR3, CXCR4, and CXCR6/STRL33. The level of each chemokine or chemokine receptor was calculated relative to the level of ubiquitin expressed in that sample. Of all the genes tested, CCL18 was the most strikingly and consistently increased chemokine in lung parenchyma from hypersensitivity pneumonitis and idiopathic pulmonary fibrosis patients, but not in lung parenchyma from either control or asthmatic lungs. Two of the control lung samples and one asthmatic sample had high levels of CCL18. The levels were 1556, 1502, and 1393. There is nothing remarkable in the clinical histories of these three patients that may indicate underlying infection or lung disease. Since the receptor for CCL18 has not been identified, we were unable to test the samples for expression of its receptor.

XI. Localization of CCL18 mRNA and Immunoreactive Protein in Lung Tissue

To examine which cells in the lung of HP patients made CCL18, we performed both in situ hybridization and immunohistochemical studies on tissue sections from HP biopsies. By in situ hybridization CCL18 transcript was found in reactive type 2 alveolar epithelial cells and in isolated interstitial cells, mainly macrophages. CCL18 expression was primarily observed in areas of more severe lung inflammation. Control tissues hybridized with antisense CCL18 probe were negative, as were HP tissues hybridized with the sense probe.

The expression pattern of the immunoreactive protein paralleled the mRNA observations. Interstitial macrophages strongly stained with CCL18 monoclonal antibody, while reactive alveolar epithelial cells produced immunoreactive protein. Immunohistochemical staining for CCL18 was negative in normal lungs. HP tissue samples incubated with non-immune sera were also negative.

CCL18 mRNA expression was also significantly increased in lung tissue from IPF patients, although to a lesser degree than in HP. We also performed in situ hybridization and immunohistochemical studies on tissue sections from IPF patients to determine whether the same cell types produce CCL18 in this second interstitial lung disease. CCL18 mRNA was produced by reactive alveolar epithelial cells in an IPF patient, while sections hybridized with the sense control showed no staining. Immunohistochemical staining with anti-CCL18 monoclonal antibody identified interstitial macrophages as a source of this protein. Tissue sections from control lungs showed no immunoreactive protein. These findings indicate that the same cell types produce CCL18 in both diseases.

XII. Correlation of Tissue CCL18 in Tissue with Lymphocytes in BAL

CCL18 has been shown to chemoattract naïve T cells. In addition, it was identified in areas of severe lung inflammation by in situ hybridization and immunohistochemical staining in both HP and IPF. Therefore, we hypothesized that there may be a correlation between the level of CCL18 in lung tissue and the level of lymphocytes in the BAL fluid of these patients. The data shows a significant correlation between these two parameters. In addition, the patients with the highest levels of CCL18 had acute rather than chronic HP. These patients also exhibit the highest numbers of lymphocytes in their BAL. This observation supports the hypothesis that CCL18 expression in the lung results mediates the recruitment of T lymphocytes in acute lung disease.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A method of diagnosing or evaluating a pulmonary condition, said method comprising evaluating expression of CCL18.

2. The method of claim 1, wherein said pulmonary condition is inflammatory or asthmatic.

3. The method of claim 1, wherein said pulmonary condition is hypersensitivity pneumonitis, idiopathic pulmonary fibrosis, or asthma.

4. The method of claim 1, wherein said evaluation is of bronchoalveolar lavage (BAL) or tissue biopsy.

5. The method of claim 4, wherein said evaluation is of nucleic acid or protein.

6. The method of claim 4, wherein said evaluation is histological.

7. The method of claim 1, in combination with another diagnostic for said pulmonary condition.

8. A method of treating an inflammatory lung condition, said method comprising blocking signaling mediated by CCL18.

9. The method of claim 8, wherein said lung condition exhibits signs or symptoms of hypersensitivity pneumonitis, idiopathic pulmonary fibrosis, or asthma.

10. The method of claim 8, wherein said blocking is by administering an antibody which neutralizes CCL18 signaling.

11. The method of claim 8, wherein said blocking is by receptor desensitization.

12. The method of claim 8, in combination with another treatment for said lung condition.
